(19) United States
(12) Patent Application Publication
(10) Pub. No.: US 2005/0165440 A1
(43) Pub. Date: Jul. 28, 2005

(54) System for treating obesity and implant for a system of this type

(76) Inventors: Richard Cancel, La Garde (FR); Richard Wallace, Gonfaron (FR); Gerard Sassi, Toulon (FR); Daniel Grepillat, Sevrier (FR)

Correspondence Address: IP GROUP OF DLA PIPER RUDNICK GRAY CARY US LLP, 1650 MARKET ST, SUITE 4900, PHILADELPHIA, PA, 19103, US

(21) Appl. No.: 11/008,433

(22) Filed: Dec. 9, 2004

Related U.S. Application Data
(63) Continuation of application No. PCT/FR03/01505, filed on May 16, 2003.

(30) Foreign Application Priority Data
Jun. 13, 2002 (FR) 02/07283
Feb. 11, 2003 (FR) 03/01619

(51) Int. Cl. A61N 1/08

(52) U.S. Cl. 606/191

(57) ABSTRACT

A system for treating obesity in a patient including a gastric implant to control alimentary absorption, the gastric implant including a ring sized and shaped to fit around an esophagus of the patient and a motorized controller to adjust extension of the ring, and an induction coil to power the motorized controller, wherein the induction coil is integrated in the implant; and a control equipment unit including a device to adjust emission power of the induction coil; wherein the implant further includes an electronic circuit delivering a response signal to a command transmitted by the control equipment unit.

### RELATED APPLICATION

This is a continuation of International Application No. PCT/FR03/01505, with an international filing date of May 16, 2003 (WO 03/105732, published Dec. 24, 2003), which is based on French Patent Application Nos. 02/07283, filed Jun. 13, 2002, and 03/01619, filed Feb. 11, 2003.

### FIELD OF THE INVENTION

This invention pertains to a system for treating obesity including a motorized gastric implant and a control equipment unit intended for a medical operator.

### BACKGROUND

WO 01/12108 describes a gastric implant controlled by a power transmission means providing a wireless transmission of power in a first form from the exterior of the body of a patient. An implanted medical device can operate in response to power in a second form different from the power of the first form. An implanted power transmission means transforms the power of the first form, transmitted in a wireless manner by the power transmission means, into power of the second form intended to be used in the control and operating of the medical device.

WO 00/15158 describes another constriction gastric implant, at least partially implantable in the human or animal body comprising a constriction member forming a ring in its operating configuration. This constriction member comprises a flexible band, both ends of which are adjacent to each other in the operating configuration, as well as a means for actuating the constriction member. One end of the flexible band comprises a tractable element enabling displacement of the end in relation to the other end generating a radial deformation of the constriction member.

WO 01/12076 A1 describes a device for the treatment of gastric burning sensations and gastroesophogeal reflux which comprises a controllable constriction device that can be implanted in a patient or animal, the device being intended to come into contact with the stomach close to the cardia or to come into contact with the esophagus in a manner to form a constriction in the alimentary canal. A power emitter enabling a wireless emission of power of a first form originating from the exterior of the patient is provided. This constriction device can be made to operate by reaction of power of a second form different from the power of the first form, delivered in a manner to vary the constriction in the alimentary canal. An implantable power transformer is used to transform the power of the first form emitted by the power emission organ into power of the second form.

The implementation of such implants creates difficulties linked to the electromagnetic power necessary for the good operation of the implant. It is in fact important that the induction power be sufficient to ensure good operation of the motor but, if the power is excessive, it can cause heating which can become harmful.

This difficulty is increased by the fact that the positioning of the implant varies from one patient to another, and that the relative orientation as well as the distance between the antennas of the implant and the control equipment unit has a major impact on the transmitted power. One solution consists of separating the induction coil from the implant to allow positioning of the coil close to the patient's skin. Although this certainly reduces power transmission problems, it involves a more intensive surgical intervention.

It would therefore be advantageous to avoid such a surgical intervention and make it unnecessary for the patient to have an antenna under the skin, the antenna being furthermore required to be physically linked to the implant. It would also be advantageous to resolve the problem of the adequacy of the transmitted power and to increase the safety of use of the gastric rings.

### SUMMARY OF THE INVENTION

This invention relates to a system for treating obesity in a patient including a gastric implant to control alimentary absorption, the gastric implant including a ring sized and shaped to fit around an esophagus of the patient and a motorized controller to adjust extension of the ring, and an induction coil to power the motorized controller, wherein the induction coil is integrated in the implant; and a control equipment unit including a device to adjust emission power of the induction coil, wherein the implant further includes an electronic circuit delivering a response signal to a command transmitted by the control equipment unit.

This invention also relates to a gastric implant for a patient including a ring sized and shaped to fit around an esophagus of the patient, a motorized controller to adjust the ring, an induction coil for contactless powering of the motorized controller integrated in the implant, and an electronic circuit delivering a response signal to a command transmitted by a control equipment unit.

### BRIEF DESCRIPTION OF THE DRAWINGS

Better comprehension of the invention will be obtained from the description below with reference to the attached drawings pertaining to a nonlimitative example of implantation in which:

FIG. 1 is a sectional view of an implant according to aspects of the invention;

FIG. 2 is a block diagram of the control equipment unit;

FIG. 3 is a block diagram of the electronic circuits of the implant;

FIG. 4 is a schematic representation of the operating algorithm of the control equipment unit;

FIG. 5 is a schematic representation of the operating algorithm of the implant; and

FIG. 6 is a block diagram of another aspect of the control equipment unit (command console—transmitter/receiver).

### DETAILED DESCRIPTION

The invention pertains, in its most general sense, to a system for treating obesity comprising a gastric implant to control alimentary absorption and a control equipment unit, the gastric implant comprising a ring designed to fit around the esophagus and a motorized controller for adjusting the extension of the ring, as well as an induction coil for the wireless powering of the motorized controller, wherein the induction coil of the implant is integrated in the implant and the control equipment unit comprises means for adjusting the emission power and the implant comprises an electronic circuit delivering a response signal to a command transmitted by the control equipment unit.

The implant advantageously comprises:

a memory for recording digital identification data of the implant (date of manufacture, series number, etc.);

and/or a memory for recording digital identification data of the patient (date of implantation, identifier, etc.);

and/or a memory for recording digital data corresponding to historical events (date of modification, position);

and/or a memory for recording the absolute position of the implant;

and/or a memory for recording displacement limits of the ring and a means for preventing command of the motor in the case of receiving commands which would lead to going beyond the limits.

According to one preferred aspect, the control equipment unit comprises means for receiving information recorded in the memory of an implant and a memory for recording, e.g., historical data, comprising the identifier of each of the controlled implants and the date of modification of each of the implants.

The control equipment unit advantageously comprises a mobile antenna and a means for generation of a digital reference screen for the step of adjustment of the emission power prior to the step of commanding an implanted ring. The control equipment unit preferably comprises a circuit for modulation of the amplitude of the feed voltage of the mobile antenna.

According to one particular aspect, the implant comprises a time delay activating the vigilance mode after a predetermined period without command signals. According to another aspect, the system comprises a test function commanding the displacement of the implant, i.e., the closing then the opening of a position.

The control equipment unit preferably comprises a calculator to create a command signal which is a function of a position input by an operator and the control information stemming from the implant to be commanded. According to one preferred aspect, the control equipment unit is capable of generating a secure signal for the displacement of one step of the implant motor.

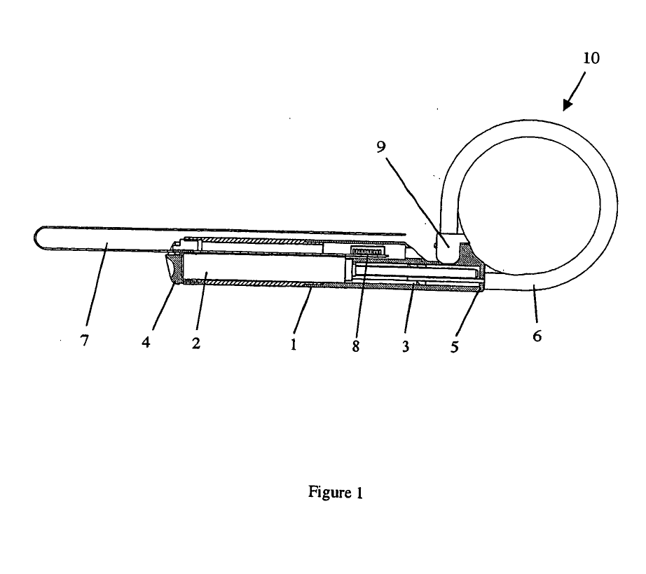

Turning now to the Drawings, the implant shown in FIG. 1 comprises a case (1) of a section of about 10 mm up to about 20 mm to allow housing of the antenna in the case and to enable implantation by laparoscopy. Case (1) is closed by a cap (4) that incorporates an electronic circuit (8) and a brushless motor (2) driving a threaded coupling (3) capable of translational displacement in a tubular lip (5). This threaded coupling (3) extends one of the ends of a band (6) forming the gastric ring (10). This ring has a deployed length of about 85 millimeters. The maximum perimeter is preferably 85 millimeters. The opposite end of the threaded coupling (3) has a hook for attachment to the body of the case (1) after implantation around the esophagus. The implant further comprises an induction coil comprising a support of rectangular form having conductive windings.

The course of the threaded coupling (3) is about 15 to about 25 millimeters. The length of the case is about 70 to about 85 millimeters. The length of the induction coil is about 67 to about 82 millimeters. The displacement rate is about 12 to about 25 mm per minute with a force of about 8 to about 12 newton. The course is divided into 8 positions distributed linearly with a precision of approximately 10%. According to one aspect, the motor comprises an encoder formed, e.g., by a Hall sensor detecting angular displacement and transmitting a relative displacement signal to the electronic circuit of the implant. According to another aspect, the control electronics reconstitute the angular position of the motor then, by counting, the position of the threaded coupling.

The electronic circuit integrated in the implant comprises a nonvolatile random access memory for recording before implantation information not modifiable after the locking of each of them, such as:
the series number of the implant;
the patient's identifier;
the date of manufacture;
the date when initially put into service;
as well as information that can be modified after implantation:
the date of the last intervention;
historical information on the last N interventions, e.g., the last 8 interventions;
the number of interventions since first being put into service.

The implant is positioned such that the ring surrounds the esophagus and not the stomach. Its operation is controlled by a control equipment unit comprising a command console and a mobile antenna that can be positioned close to or preferably on the patient's abdomen with a slight possibility of angular orientation.

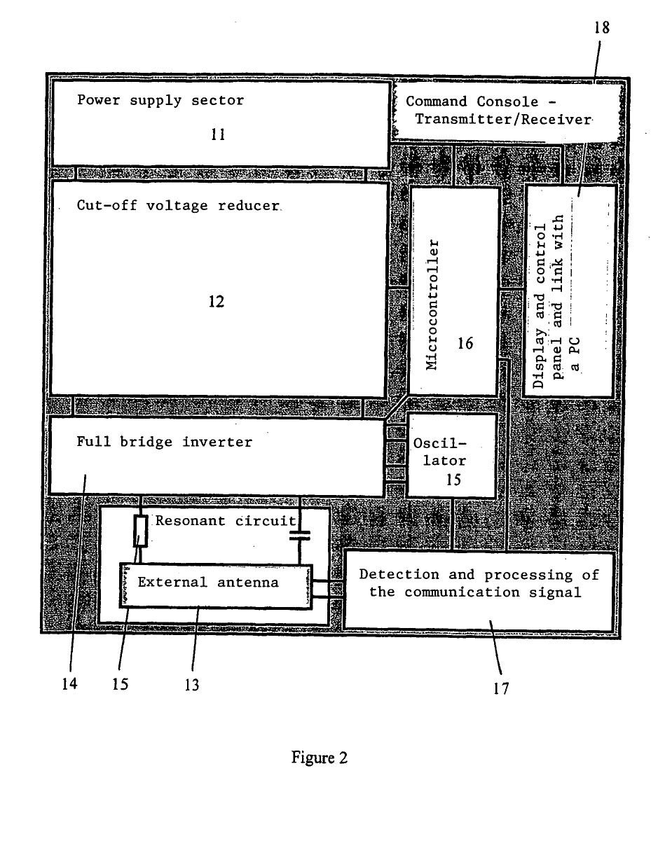

A block diagram of the control equipment unit is shown in FIG. 2. It comprises a cut-off power supply (11, 12) with a cut-off voltage reducer circuit (12). The antenna (13) is coupled to a full bridge inverter (14) by a tuned circuit (15). The power transmitted to the antenna (13) is modulated or regulated in amplitude by a step-down device (12) controlled by a microcontroller (16). According to one aspect, the microcontroller (16) communicates with an input/output interface (18) controlling a display panel and/or providing the link with a microcomputer. According to another aspect, the microcontroller (16) communicates with a microcomputer which is in the console.

The equipment unit further comprises a circuit (17) to detect and process the signal stemming from the implant. A time-delay circuit interrupts the powering of the antenna after a predetermined cumulative duration of emission so as to avoid excessive heating.

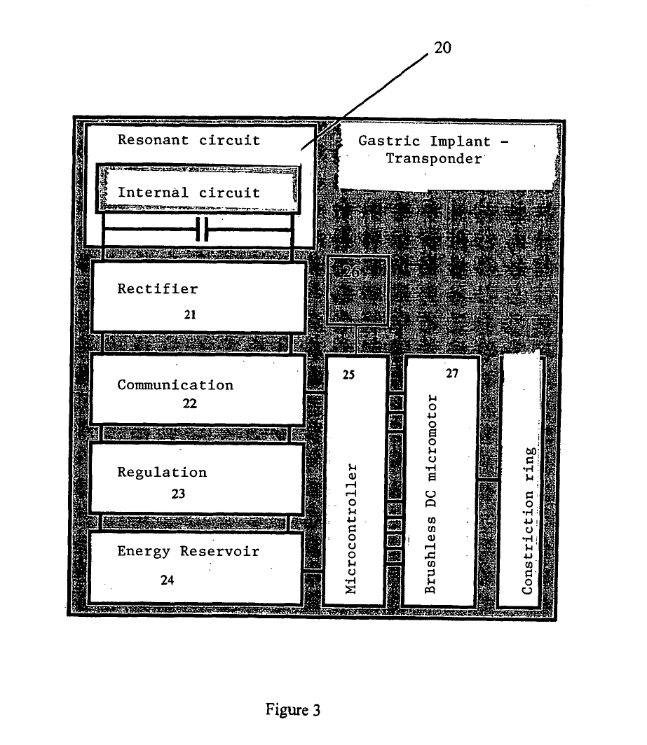

The block diagram of the electronic circuit of the implant is shown in FIG. 3. It comprises an induction coil (20) linked by a rectifier to a communication circuit (22). According to one aspect, a power supply (24) in the form of a buffer battery or a condenser or any equivalent means powers the electronic circuits by the intermediary of a regulation circuit (23). According to another aspect, the coil (20) powers the electronic circuits. A microcontroller (25) controls the brushless motor (26) as well as the memories integrated in the microcontroller (25).

The electronic circuit of the implant controls the following functions:
Generation of a specific screen or localization screen for the localization of the control equipment unit during the positioning of the antenna;
Management of the memories, and notably of the locking of information recorded prior to implantation by modification of the state of a flag preventing new writing in the corresponding registers;
Transmitting of data consisting of the generation of messages under the action of a command transmitted by the control equipment unit;
Displacement of the threaded coupling as a function of the instruction emitted by the control equipment unit. Displacement is controlled without sensors, with verification of the displacement by the implant at each position before any new displacement command;
Repositioning of the ring: this function is activated by the control equipment unit under the operator's control when there is incertitude about the effective position of the motor in relation to the recorded information. The control equipment unit sends a message commanding the displacement at the implant motor's stop, in maximal and minimal extension, so as to return to zero the position information and calculate the limited positions.

The implant circuit also generates alarm signals in the case of malfunction:
Detection of blockage: this signal is generated when a displacement command signal is detected and the motor remains inactive; this blockage is detected according to a first aspect because no modification of the signal is provided by the angular position detector of the motor and according to a second aspect in the absence of sensor as a function of the fact that the motor does not start up or slows down to a major degree. The error signal is recorded in a memory of the implant. In a first aspect, the system generates a return command. In a second aspect, the implant returns to its previous position.

The control equipment unit has multiple possible states:
Stop: the electric power is interrupted and the electronic circuits are at rest. The antenna does not emit any radiation;
Waiting: the power is activated and the circuits are powered, the antenna does not emit any radiation;
Normal use: the antenna is powered, the implant is identified;
Use/localization: the antenna is powered and the equipment unit is in phase of adjustment of the emission power by periodic incrementing of the power and measurement of the implant's response, with repositioning of the antenna in order to optimize the transmitted power;
Use/command: the power is set and the equipment unit transmits the command information to the implant;
Use/alarm: the implant or the equipment unit generates an alarm signal.

A timer activates the waiting mode after a use of a duration greater than a predetermined value.

Similarly, the implant has different operating states:
Waiting: the default state. The electronic circuits and the motor are not powered;
Localization: the implant detects an emission and dialogues with the equipment unit for the purpose of optimizing the emission power. The goal is to set the emission power at a minimum level required for transmitting sufficient power for the operating of the electronic circuits then the brushless motor, with an unequivocal detection of the command signals modulating the amplitude of the voltage applied to the antenna of the control equipment unit;
Ready: the implant is ready to receive command signals;
Alarm: the implant generates an alarm signal in response to an operating anomaly;
Vigilance: the implant does not respond subsequent to an excessively long period of inactivity of the control equipment unit.

The description below pertains to an advantageous mode of operating a system according to aspects of the invention and specifies several important functionalities.

The control equipment unit first of all emits a command signal for identification of the medical operator. For this purpose, it comprises a procedure for inputting a password and verification, e.g., by means of a cryptologic algorithm. The verification of this procedure opens a session which ends after a determined period of inactivity.

The management of the passwords and the users, and especially of new users, is performed by means of a procedure protected by an access master.

The creation of a new patient is performed from the opening of a session of a user referenced at a higher level. The control equipment unit comprises a keyboard for inputting the specific information regarding a new patient such as an identifier and for the recording of this information in the memory of a new implant. These data are locked upon the first activation of the implant's motor. The control unit comprises for this purpose an algorithm neutralizing the access in writing of the memory zone intended to receive the information pertaining to the identity of the patient in response to a signal, e.g., after the first request for displacement of the motor.

Prior to implantation, the system allows verification of good operating order. For this purpose, the control equipment unit transmits instructions to confirm the rotation of the motor first in one direction and then in the other direction in a single step.

The command of an implant comprises a step of positioning the antenna by the practitioner. He displaces the antenna in relation to the patient's body until obtaining, for a given power, the best response signal. The power is then diminished incrementally until reaching the minimum power compatible with a satisfactory powering of the implant.

After displaying the data stored in memory in the implant, the practitioner can then command displacement of the motor, either by calculation of a target displacement or by a step-by-step modification of the positioning of the motor.

In the first case, the control equipment unit inputs the actual position of the implant by reading the implant's position memory then calculating the number of positions required for the target position. The equipment unit then modulates the command signal to the implant to command the displacement of the implant's motor according to a secure protocol.

Another mode of use comprises sending a command signal to provide for the displacement of one step in one direction or in the other direction.

Each displacement is performed on a step-by-step basis to ensure the correct performance of the displacements.

This mode makes it possible to increase or reduce the section of the gastric ring.

Each displacement performed by the implant is recorded in the implant's memory to save the history of the modification. Only the last modification is time stamped.

The system moreover comprises an urgent opening procedure allowing commanding of the maximal opening of the ring without passing through a prior identification step.

The operating of the implant comprises different procedures. The initialization procedure consists of generating a specific screen that can be detected by the control equipment unit during the phase of adjustment of the power applied to the antenna.

This procedure is triggered by the detection by the implant of a modulated signal specific to the control equipment unit, so as notably to limit the risk of interaction with other induction devices. It is interrupted, e.g., upon emission of a confirmation signal by the control unit.

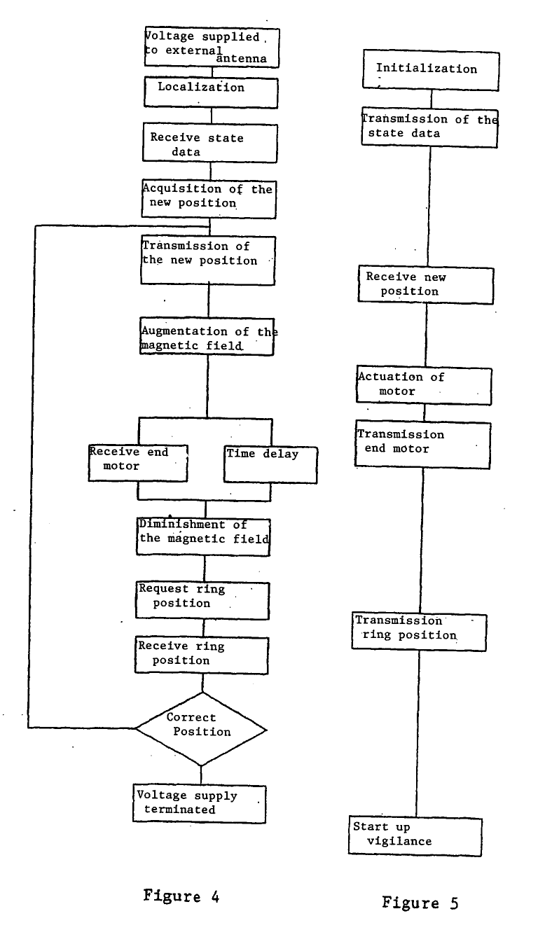

The invention was described above with reference to a preferred example of implementation. Nonlimiting examples of operating algorithms of the control equipment unit and the implant are shown respectively in FIGS. 4 and 5.

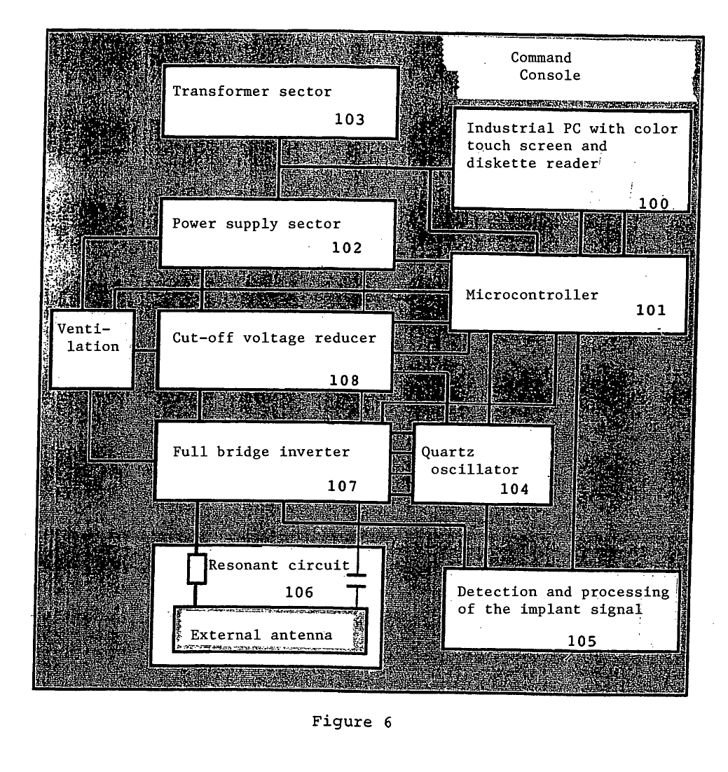

FIG. 6 shows a block diagram of a variant of implementation of the control equipment unit (command console—transmitter/receiver). The command console of this aspect is constituted of a control card and an industrial computer (100) with a touch screen.

The touch screen enables creation of contextual menus by a software application loaded in the random access memory of the computer. This application program activates the tactile zones corresponding solely to the authorized functions, taking into account the state of the system, and prevents all other actions. This solution makes it possible to increase the security of use of the system.

Certain security functions such as the management of the passwords are provided not by the computer (100) but by the microcontroller (101) of the control card of the gastric ring.

The computer provides the man-machine interface functions while the implant command functions are provided by the electronic circuits of the control card. A peripheral device, e.g., a diskette reader makes it possible to store and/or process the data in memory.

In addition to the microcontroller (101), this control card comprises a power supply (102) comprising a sector transformer (103), a quartz oscillator (104) delivering a clock signal and a circuit (105) for the detection and processing of the signals exchanged with the implant.

The power supply and communication with the implant is provided by an antenna (106) powered by a full bridge inverter (107). According to this aspect, the feed voltage of the antenna (106) is reduced by a cut-off voltage converter (108). This makes it possible to limit the emission power to that strictly required for the correct operation of the implant. The antenna of the implant is formed by a plastic frame around which is coiled a winding, e.g., spires of enameled copper wire. The frame is integrated in the implant which enhances the reliability of the construction of the implant. According to one aspect, one uses a thin antenna at the exterior of the case which antenna must be inserted in the patient's body in coiled up form. According to another aspect, the antenna is placed in the case: this eliminates the risk of failure due to rupture of the thin antenna after insertion.

The communication frequency is set at 115.2 kHz to 120.6 kHz so as to enable an optimal transmission of power at a distance of less than 10 centimeters.

1. A system for treating obesity in a patient comprising:
a gastric implant to control alimentary absorption, the gastric implant comprising a ring sized and shaped to fit around an esophagus of the patient and a motorized controller to adjust extension of the ring, and an induction coil to power the motorized controller, wherein the induction coil is integrated in the implant; and
a control equipment unit comprising a device to adjust emission power of the induction coil;
wherein the implant further comprises an electronic circuit delivering a response signal to a command transmitted by the control equipment unit.

2. The system according to claim 1, the electronic circuit comprising a memory for recording digital identification data of the implant.

3. The system according to claim 1, the electronic circuit comprising a memory for recording digital identification data of the patient.

4. The system according to claim 1, the electronic circuit comprising a memory for recording digital data corresponding to historical information.

5. The system according to claim 1, wherein the implant comprises a memory for recording an absolute position of the ring.

6. The system according to claim 1, wherein the implant comprises a memory for recording displacement limits of the ring, and a means for preventing command of the motor in the case of receiving commands that would lead to going beyond desired limits.

7. The system according to claim 1, wherein the control equipment unit comprises means for receiving information recorded in the memory of an implant and a memory for recording the historical information, comprising the identifier of each controlled implant and the date of modification of each implant.

8. The system according to claim 1, wherein the control equipment unit comprises a mobile antenna and a generator of a digital reference screen for adjusting emission power prior to a command step of an implanted antenna.

9. The system according to claim 1, wherein the control equipment unit comprises a circuit that regulates amplitude of a feed voltage of a mobile antenna.

10. The system according to claim 1, wherein the implant comprises a time-delay circuit activating a vigilance mode after a predetermined period without command signals.

11. The system according to claim 1, further comprising a test function commanding closing of a position, then opening of a position of the implant and the control equipment unit comprising test function deactivation information.

12. The system according to claim 1, wherein the control equipment unit comprises a calculator that creates a command signal which is a function of a position input by an operator and command information stemming from the implant to be controlled.

13. The system according to claim 1, wherein the control equipment unit is capable of generating a secure signal to displace one step of an implant motor.

14. The system according to claim 1, wherein an induction coil of the implant comprises a frame comprising a winding, the frame being integrated in the interior of the implant.

15. The system according to claim 1, wherein the command circuit comprises a cut-off voltage converter inserted between a power supply and a full bridge inverter making it possible to limit the emission power.

16. A gastric implant for a patient comprising a ring sized and shaped to fit around an esophagus of the patient, a motorized controller to adjust the ring, an induction coil for contactless powering of the motorized controller integrated in the implant; and an electronic circuit delivering a response signal to a command transmitted by a control equipment unit.

17. The gastric implant according to claim 16, wherein the induction coil is implemented by a winding around a frame, the frame being integrated in the implant.
